(19) United States
(12) Patent Application Publication
(10) Pub. No.: US 2023/0228766 A1
(43) Pub. Date: Jul. 20, 2023

(54) SERUM BIOMARKERS FOR PREDICTING AND PREVENTING HEMORRHAGIC STROKE

(71) Applicant: Texas Tech University System, Lubbock, TX (US)

(72) Inventors: Jun Zhang, Lubbock, TX (US); Johnathan Abou-Fadel, Lubbock, TX (US)

(73) Assignee: Texas Tech University System, Lubbock, TX (US)

(21) Appl. No.: 18/007,727

(22) PCT Filed: Jun. 4, 2021
(86) PCT No.: PCT/US2021/035907

§ 371 Date: Dec. 1, 2022

Related U.S. Application Data
(60) Provisional application No. 63/034,787, filed on Jun. 4, 2020.

(51) Int. Cl. G01N 33/68; G16H 50/30

(52) U.S. Cl. CPC G01N 33/6893; G01N 33/6869; G16H 50/30; G01N 33/6827; G01N 2800/60; G01N 2800/50

(57) ABSTRACT

The present disclosure pertains to predicting an onset of a cerebral hemorrhagic event in a subject by testing a blood sample of the subject for the presence or absence of one or more indicators; and correlating the presence or absence of the one or more indicators to the subjects risk for suffering from the cerebral hemorrhagic event, where the presence of the one or more indicators is correlated to an increased risk in the subject for suffering from the cerebral hemorrhagic event and where the absence of the one or more indicators is correlated to a decreased risk in the subject for suffering from the cerebral hemorrhagic event. The methods of the present disclosure may also include implementing a therapeutic decision in order to prevent the cerebral hemorrhagic event. The present disclosure also pertains to kits for use in predicting an onset of a cerebral hemorrhagic event in a subject.

### CROSS-REFERENCE TO RELATED APPLICATIONS

This application claims priority to U.S. Provisional Patent Application No. 63/034,787 filed on Jun. 4, 2020. The entirety of the aforementioned application is incorporated herein by reference.

### BACKGROUND

A need exists for non-invasive methods for predicting and preventing cerebral hemorrhagic events. Numerous embodiments of the present disclosure address the aforementioned need.

### SUMMARY

In some embodiments, the present disclosure pertains to methods of predicting an onset of a cerebral hemorrhagic event in a subject. In some embodiments, the methods of the present disclosure include: testing a blood sample of the subject for the presence or absence of one or more indicators; and correlating the presence or absence of the one or more indicators to the subject's risk for suffering from the cerebral hemorrhagic event, where the presence of the one or more indicators is correlated to an increased risk in the subject for suffering from the cerebral hemorrhagic event, and where the absence of the one or more indicators is correlated to a decreased risk in the subject for suffering from the cerebral hemorrhagic event. In additional embodiments, the present disclosure also includes a step of implementing a therapeutic decision in order to prevent the cerebral hemorrhagic event.

Further embodiments of the present disclosure pertain to kits for use in predicting an onset of a cerebral hemorrhagic event in a subject. In some embodiments, the kit includes a testing platform for testing a blood sample of the subject for the presence or absence of one or more indicators of the present disclosure. In some embodiments, the kits of the present disclosure also include a platform for correlating the presence or absence of the one or more indicators to the subject's risk for suffering from a cerebral hemorrhagic event.

In some embodiments, the one or more indicators include: an increased level of progesterone (PRG) relative to a reference blood sample, a decreased level of Albumin relative to a reference blood sample, an increased level of Serpin A6 relative to a reference blood sample, a decreased level of IL-6 relative to a reference blood sample, a decreased level of IL-12 relative to a reference blood sample, or combinations thereof. In some embodiments, the one or more indicators include two or more, three or more, four or more, or all of the aforementioned indicators.

### DESCRIPTION OF THE DRAWINGS

FIG. 1 illustrates a method of predicting an onset of a cerebral hemorrhagic event in a subject.

FIG. 2 illustrates cerebral cavernous malformations (CCM) signal complex (CSC) as a master regulator of homeostasis of progesterone (PRG) and synergistic effects of PRG and its antagonist, mifepristone (MIF), on the two types of PRG receptors (nPRs/mPRs). Red color indicates the effect from the CSC onto PRG and both classic and nonclassic PRG receptors (nPRs, mPRs). The lower lines indicate effect from PRG onto its PRG receptors (thicker lower lines indicate strong effects, and dotted lines suggest weak effects). The upper lines indicate the effects of PRG receptors onto the CSC. + Symbol indicates enhancement, while − symbol demonstrates an inhibitory effect.

FIG. 3 illustrates that PRG and MIF can work synergistically to inhibit protein expression of CCM 1/3, two key components within CSC, in endothelial cells (ECs). In particular, the results indicate that the combination of PRG and MIF synergistically enhances their inhibitory effects on protein expression levels of CCM 1/3 in nPR negative(−) ECs: human dermal microvascular endothelial cells (HDMVEC), human brain microvascular endothelial cells (HBMVECs), and rat brain microvascular endothelial cells (RBMVEC); and nPR positive(+) ECs: human umbilical vein endothelial cells (HUVEC), compared to MIF only or vehicle controls (VEH).

FIGS. 4A and 4B illustrate the effects of combined sex hormone (PRG+MIF) actions on the in-vitro permeability of two types of endothelial cells (ECs). The in-vitro permeability of two different ECs lines, nPR(+) EAhy926 ECs derived from HUVECs (FIG. 4A), and nPR(−) RBMVECs (FIG. 4B), was measured with the passage of FITC-conjugated dextran under combined sex hormone (PRG+MIF) treatment. Although increased levels of permeability were initially observed in both ECs, the permeability of nPR(+) EAhy926 ECs is back to normal after 12 hours, while the permeability was continuously enhanced among all sex hormone treatments (PRG, MIF, PRG+MIF) of RBMVECs. Four treatments are Vehicle, PRG, MIF, and PRG+MIF sequentially.

FIGS. 5A and 5B illustrate increased blood brain barrier (BBB) permeability and negatively correlated serum level of cytokine, IL-6, under chronic exposure of excessive sex steroid on Ccm mutant mouse strains. In FIG. 5A, BBB permeability is measured by the tissue extravasation of Evans blue (EBD, Miles assay). Significantly increased BBB permeability is observed in all three Ccms mutants when compared to wild type (WT). In FIG. 5B, IL-6 is measured with an enzyme-linked immunosorbent assay (ELISA). Significantly decreased amounts of IL-6 were observed in all Ccm mutants when compared to WT. Both panels are presented in 90-day groups of (PRG+MIF) treatment.

FIG. 6 illustrates correlation functions for 5 serum biomarkers among three Ccms (1-3) mutant strains. Serum levels of PRG, Serpin A6, Albumin, IL-6, and IL-12 either were found to positively or negatively correlate with vascular permeability in BBB leakage. Binomial regression of these five biomarkers in the serum with EBD in the brain was performed, which can be utilized as prognostic biomarkers to predict the risk for hemorrhagic strokes in human CCM patients.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Cerebral cavernous malformations (CCMs), one of the most common vascular malformations, are characterized by abnormally dilated intracranial capillaries resulting in increased susceptibility to hemorrhagic stroke. As an autosomal dominant disorder with incomplete penetrance, the majority of CCMs gene mutation carriers are largely asymptomatic. However, when symptoms occur, the disease has typically reached the stage of focal hemorrhage with irreversible brain damage.

Currently, the invasive neurosurgery removal of CCM lesions is the only treatment option, despite the recurrence of symptoms after surgery. The surgical removal of CCM lesions would temporarily alleviate the health risks of CCMs. However, later invasive neurosurgeries required for additional CCMs have devastating consequences for patients.

Therefore, a need exists for non-invasive methods for predicting and preventing cerebral hemorrhagic events. Numerous embodiments of the present disclosure address the aforementioned need.

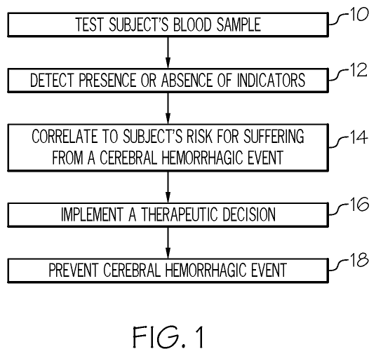

In some embodiments, the present disclosure pertains to methods of predicting an onset of a cerebral hemorrhagic event in a subject. In some embodiments illustrated in FIG. 1, the methods of the present disclosure include: testing a blood sample of the subject (step 10) for the presence or absence of one or more indicators (step 12); and correlating the presence or absence of the one or more indicators to the subject's risk for suffering from the cerebral hemorrhagic event (step 14), where the presence of the one or more indicators is correlated to an increased risk in the subject for suffering from the cerebral hemorrhagic event, and where the absence of the one or more indicators is correlated to a decreased risk in the subject for suffering from the cerebral hemorrhagic event. In additional embodiments illustrated in FIG. 1, the present disclosure also includes a step of implementing a therapeutic decision (step 16) in order to prevent the cerebral hemorrhagic event (step 18).

Further embodiments of the present disclosure pertain to kits for use in predicting an onset of a cerebral hemorrhagic event in a subject. In some embodiments, the kit includes a testing platform for testing a blood sample of the subject for the presence or absence of one or more indicators of the present disclosure. In some embodiments, the kits of the present disclosure also include a platform for correlating the presence or absence of the one or more indicators to the subject's risk for suffering from a cerebral hemorrhagic event.

As set forth in more detail herein, the methods and kits of the present disclosure can have numerous different embodiments.

### Subjects

The methods and kits of the present disclosure can be utilized to predict an onset of a cerebral hemorrhagic event in various subjects. For instance, in some embodiments, the subject is a human being. In some embodiments, the subject is a male. In some embodiments, the subject is a female. In some embodiments, the subject is a pregnant female.

In some embodiments, the subject is vulnerable to suffering from a cerebral hemorrhagic event. In some embodiments, the subject is vulnerable to suffering from a hemorrhagic stroke. In some embodiments, the subject has suffered from a hemorrhagic stroke.

### Blood Samples

The methods and kits of the present disclosure may test various types of blood samples. For instance, in some embodiments, the blood sample includes whole blood samples. In some embodiments, the blood sample includes blood plasma. In some embodiments, the blood sample is purified blood plasma. In some embodiments, the blood sample includes blood serum. In some embodiments, the blood sample is purified blood serum.

In some embodiments, the methods of the present disclosure also include a step of collecting a blood sample from a subject for testing. In some embodiments, the collected blood sample is processed before testing. For instance, in some embodiments, the blood plasma is purified and isolated from the collected blood sample for testing. In some embodiments, the blood serum is isolated from the collected blood sample for testing.

### Testing

The methods and kits of the present disclosure can utilize various tests and testing platforms for testing for the presence or absence of one or more indicators. For instance, in some embodiments, the testing includes testing for the presence or absence of a protein of the one or more indicators. In some embodiments, the testing occurs through the utilization of an enzyme-linked immunosorbent assay (ELISA). In some embodiments, the testing occurs through the utilization of an ultra-sensitive multiplex bead array assay (MBAA).

In some embodiments, the testing includes testing for the presence or absence of a messenger RNA transcript of the one or more indicators. In some embodiments, the testing occurs through reverse transcription of the messenger RNA transcripts. In some embodiments, the reverse transcription occurs through the utilization of quantitative real-time PCR (RT-qPCR).

In some embodiments, the testing occurs in a single time period. In some embodiments, the testing occurs on a recurrent basis.

### Indicators

The methods and kits of the present disclosure can be utilized to test for the presence of various indicators. For instance, in some embodiments, the one or more indicators include: an increased level of progesterone (PRG) relative to a reference blood sample, a decreased level of Albumin relative to a reference blood sample, an increased level of Serpin A6 relative to a reference blood sample, a decreased level of IL-6 relative to a reference blood sample, a decreased level of IL-12 relative to a reference blood sample, or combinations thereof.

In some embodiments, the one or more indicators include two or more of the aforementioned indicators. In some embodiments, the one or more indicators include three or more of the aforementioned indicators. In some embodiments, the one or more indicators include four or more of the aforementioned indicators. In some embodiments, the one or more indicators include an increased level of progesterone (PRG) relative to the reference blood sample, a decreased level of Albumin relative to the reference blood sample, an increased level of Serpin A6 relative to the reference blood sample, a decreased level of IL-6 relative to the reference blood sample, and a decreased level of IL-12 relative to the reference blood sample.

In some embodiments, the increased level of PRG is represented by an increase of at least 3.5 pg/ml relative to the reference blood sample. In some embodiments, the decreased level of Albumin is represented by a decrease of at least 1,385 pg/ml relative to the reference blood sample. In some embodiments, the increased level of Serpin A6 is represented by an increase of at least 13.4 pg/ml relative to the reference blood sample. In some embodiments, the decreased level of IL-6 is represented by a decrease of at least 40 pg/ml relative to the reference blood sample. In some embodiments, the decreased level of IL-12 is represented by a decrease of at least 50 pg/ml relative to the reference blood sample.

The methods and kits of the present disclosure may also utilize various reference blood samples. For instance, in some embodiments, the reference blood sample is obtained from one or more subjects that have not suffered from cerebral hemorrhagic events. In some embodiments, the reference blood sample is obtained from one or more subjects that do not contain CCMs gene mutations. In some embodiments, the reference blood samples do not contain gene mutations in the following CCMs genes: Ccm1, Ccm2, and Ccm3.

### Correlating

The methods and kits of the present disclosure may utilize various methods to correlate the presence or absence of the one or more indicators to a subject's risk for suffering from a cerebral hemorrhagic event. For instance, in some embodiments, the correlating occurs manually. In some embodiments, the correlating occurs through the utilization of a software. In some embodiments, the software includes a machine-learning algorithm that correlates the presence or absence of the one or more indicators to the subject's risk for suffering from the cerebral hemorrhagic event. In some embodiments, the machine-learning algorithms are regularly upgraded or adjusted to accommodate for various factors affecting the correlation of the presence or absence of one or more indicators to a subject's risk for suffering from a cerebral hemorrhagic event.

In some embodiments, the subject's risk for suffering from a cerebral hemorrhagic event is characterized by a likelihood of progression of the cerebral hemorrhagic event. In some embodiments, the subject's risk for suffering from a cerebral hemorrhagic event is characterized by a likelihood of recurrence of the cerebral hemorrhagic event. In some embodiments, the subject's risk for suffering from a cerebral hemorrhagic event is characterized by a likelihood of regression of the cerebral hemorrhagic event.

### Prevention of Cerebral Hemorrhagic Events

In some embodiments, the methods of the present disclosure also include a step of implementing a therapeutic decision to prevent a cerebral hemorrhagic event. In some embodiments, the therapeutic decision includes administering to the subject a therapeutic composition for preventing the hemorrhagic event.

In some embodiments, the therapeutic decision includes increasing Albumin levels in a subject's blood. In some embodiments, the therapeutic decision includes decreasing PRG levels in a subject's blood. In some embodiments, the therapeutic decision includes decreasing ratios of PRG and estrogen (EST) in a subject's blood. In some embodiments, the therapeutic decision includes decreasing Serpin A6 levels in a subject's blood. In some embodiments, the therapeutic decision includes increasing IL-6 levels in a subject's blood. In some embodiments, the therapeutic decision includes increasing IL-12 levels in a subject's blood.

In some embodiments, the therapeutic decision includes evaluating the subject's brain for lesions. In some embodiments, the lesions are associated with cerebral cavernous malformations (CCMs). In some embodiments, the evaluation occurs by a method that includes, without limitation, neuroimaging, magnetic resonance imaging (MRI), pathological examinations, retinal vascular examinations, or combinations thereof.

In some embodiments, the therapeutic decision includes removal of identified lesions. In some embodiments, the lesions are removed surgically.

### Cerebral Hemorrhagic Events

The methods and kits of the present disclosure can be utilized to predict the onset of numerous cerebral hemorrhagic events. For instance, in some embodiments, the cerebral hemorrhagic event includes a hemorrhagic stroke. In some embodiments, the cerebral hemorrhagic event includes cerebral hemorrhage.

### Kits

As set forth previously, further embodiments of the present disclosure pertain to kits for use in predicting an onset of a cerebral hemorrhagic event in a subject. The kits of the present disclosure generally include a testing platform for testing a blood sample of the subject for the presence or absence of one or more indicators of the present disclosure.

In some embodiments, the testing platform includes a testing platform for testing for the presence or absence of a protein of the one or more indicators. In some embodiments, the testing platform includes an enzyme-linked immunosorbent assay (ELISA). In some embodiments, the testing platform includes an ultra-sensitive multiplex bead array assay (MBAA). In some embodiments, the testing platform includes a testing platform for testing for the presence or absence of a messenger RNA transcript of the one or more indicators.

In some embodiments, the kits of the present disclosure also include a platform for correlating the presence or absence of the one or more indicators to a subject's risk for suffering from a cerebral hemorrhagic event, where the presence of the one or more indicators is correlated to an increased risk in the subject for suffering from the cerebral hemorrhagic event, and where the absence of the one or more indicators is correlated to a decreased risk for suffering from the cerebral hemorrhagic event.

In some embodiments, the correlating platform includes a software. In some embodiments, the software includes a machine learning algorithm that correlates the presence or absence of the one or more indicators to a subject's risk for suffering from a cerebral hemorrhagic event.

### Applications and Advantages

The methods and kits of the present disclosure can have numerous applications and advantages. For instance, in some embodiments, the methods and kits of the present disclosure represent the first set of blood prognostic and monitoring biomarkers to predict the risk of hemorrhagic stroke, which not only has great potential for stroke prevention but also provides a revolutionary new paradigm for hemorrhagic stroke management.

### Additional Embodiments

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. Serum Etiological Biomarkers Associated with Disruption of the Blood-Brain Barrier (BBB)

Cerebral cavernous malformations (CCMs) is a neurological disorder characterized by abnormally dilated intracranial capillaries in the brain, resulting in increased susceptibility to hemorrhagic stroke. CCMs is an autosomal dominant condition, with three known contributing genes; CCM1, CCM2, and CCM3. These CCMs proteins have been shown to interact with each other and form the CCM signaling complex (CSC).

Due to notable incomplete penetrance, the majority of CCMs gene mutation carriers are largely asymptomatic but when symptoms occur, the disease has typically reached the stage of focal hemorrhage with irreversible brain damage. Currently, the invasively neurosurgical removal of CCM lesions is the only option for treatment, despite the recurrence of symptoms after surgery.

Therefore, there is a grave need for identifying prognostic/monitoring biomarkers as risk predictors for stroke prevention. In this Example, Applicant provides experimental data related to blood biomarkers as prognostic/monitoring tools to predict the risk of hemorrhagic stroke.

### Example 1.1 Correlation Between Progesterone and Risk of Hemorrhagic Stroke

The overall risk of stroke is similar between women and men. However, postmenopausal women are at a much greater stroke risk. Women generally bear a notable lower risk of stroke during earlier life, until reaching their middle age, doubling the risk of stroke in women 10 years post-menopause. This drastically increased stroke risk in women is caused by declined levels of circulating sex steroid hormones in the blood, especially estrogen (EST) which has been widely recognized as a beneficial factor for the integrity of vasculature in the circulatory system, through its actions on nuclear and membrane estrogen receptors (nERs/mERs), mostly on vascular smooth muscle cells and endothelial cells.

Increased stroke risk associated with altered level/composition of blood female sex hormones has been well defined under several major female physiological events, such as post-menopause, pregnancy, oral contraceptive regimens, and hormone replacement therapy. Moreover, physiological change during pregnancy, caused by the altered levels/composition of circulating female sex hormones, is a major risk factor for stroke in women. Additionally, hemorrhagic stroke is the most dominant type (up to 74%) of strokes during pregnancy, indicating the correlation between the levels/composition of circulating sex steroids and the potential risk of hemorrhagic stroke.

Blood biomarkers have been explored for the prognosis of pregnancy-associated stroke risk, suggesting the potential values of blood prognostic/monitoring biomarkers for hemorrhagic stroke. Both genders have measurable amounts of progesterone (PRG) in the bloodstream.

Blood circulating PRG is approximately 0.5 ng/ml for males, while vastly fluctuating in the range of 4.0-25 ng/ml during the luteal phase of the menstrual cycle in reproductive females. Only 2% of total blood PRG is in free, active form, which has a very short half-life (5-10 min).

Over 98% of PRG in the blood is believed to be stably stored and passively transported by blood proteins. The main PRG-binding proteins are serpin A6 (binds ˜18% PRG) and albumin, (binds ˜80% PRG). PRG binding to both proteins are physiologically inactive.

### Example 1.2. New Paradigm for Hemorrhagic Progression Events in CCMS

Hemorrhage is often rooted in defective endothelial cell junctions, and microvessel rupture is a result of compromised integrity of the blood brain barrier (BBB). Currently, two major theories for the induction of hemorrhagic CCMs are the anticoagulant vascular domain theory (AVDT) and the gut microbiota theory (GMT). In the AVDT, local increases in the endothelial cofactors that generate anticoagulant APC (activated protein C) could contribute to recurrent bleeding in CCM lesions. In the GMT, gram-negative bacterial signaling through the lipopolysaccharide (LPS)-activated innate immune receptor, Toll-like receptor 4 (TLR4), promotes hemorrhagic bleeding in both Ccm1/2 mutant mice, indicating the important roles for the gut microbiome and innate immune signaling in the pathogenesis of CCMs.

GMT focuses on the importance of gut microbiota in influencing the interaction direction through inducing inflammatory gut milieu, which leads to a systemic inflamed milieu that might exacerbate the inflammatory response in the brain, and promote detrimental effects on the BBB. However, LPS-induced Ccms hemorrhagic mice demonstrates massive bleeding, leading to lethality at the early stages of life, uncharacteristic of human CCMs.

Nonetheless, neither of the previous theories could address a key issue of gender discrepancies in the pathogenesis of CCMs, demanding further evaluation for the underlying mechanisms of hemorrhagic stroke. Although it is still under debate, female dominance in CCM patients has long been suggested, and consensus has been reached on more severe bleeding with worse neurological outcomes in females. This aggressive course of hemorrhagic lesions in females has been proposed to be consequent to endocrine influences.

Applicant's prior data demonstrated that enhanced PRG-mPRs signaling, due to perturbed homeostasis of PRG, leads to CCM hemorrhagic bleeding, in addition to evidence that long exposure to hormonal contraceptives increases the risk of cerebral venous sinus thrombosis (CVST), which is incongruent with the AVDT. Applicant's prior findings that immunosuppression caused by sex steroid actions in Ccms deficient mice is associated with CCM bleeding, also disagree with the GMT.

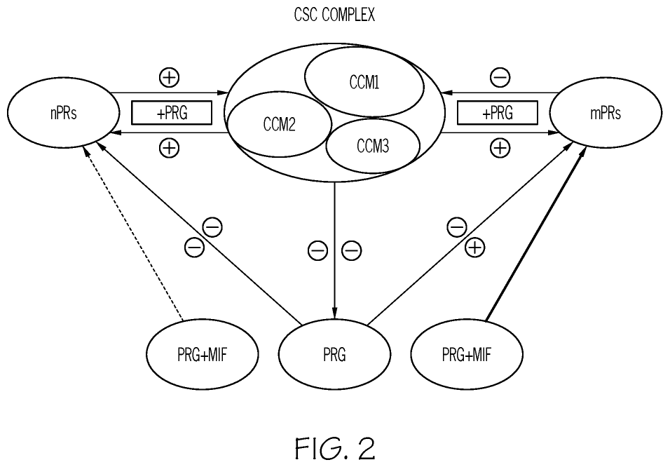

Therefore, Applicant proposed a new paradigm for the mechanisms of initiating hemorrhagic CCMs. In nPR(−) ECs, the feedback loops among the CSC-mPRs-steroid actions appear to be sensitive and perturbation of this intricate balance (FIG. 2), such as hormone therapy or hormonal contraception regimens, could result in increased risks in BBB disruption, especially for human CCMs mutant carriers. Applicant's new paradigm provides a theory that is in line with clinically observed CCM conditions.

The aforementioned paradigm is significant because current molecular genetics and neuroimaging tools are sufficient to identify mutations of CCM genes and microvascular lesions in the brain. However, due to incomplete penetrance, the majority of CCM patients do not have any knowledge of their risk for hemorrhagic strokes until they have clinical symptoms (related to hemorrhagic events). When CCM symptoms appear, neurosurgical procedures are the only option to treat CCMs patients, with a high recurrence of hemorrhagic stroke after surgery.

The surgical removal of CCM lesions would temporarily alleviate the health risks of CCMs. However, later invasive neurosurgeries required for additional CCMs have devastating consequences for patients. Therefore, there are unmet needs for identification and validation of prognostic/monitoring biomarkers to predict the risk of hemorrhagic stroke among CCM patients.

### Example 1.3. Identification of PRG as a Biomarker for Predicting Hemorrhagic Stroke

Based on Applicant's recent findings, Applicant envisions that quantitative measurement of blood biomarkers can be utilized to predict the progression of hemorrhagic events. In particular, Applicant identified five serum etiological biomarkers associated with the progression of a compromised BBB, leading to hemorrhagic events during CCM pathogenesis in three different Ccms (1-3) mutant mice models. Recent human preliminary data in two separate small pilot cohorts were in accordance with Applicant's mice results.

The CSC plays an essential role in coupling both classic and non-classic PRG signaling pathways by mediating crosstalk between them, which has put the CSC at the center stage of classic and non-classic PRG receptors mediated signaling cascades. Indeed, Applicant's data indicated that PRG (not estrogen) plays this important role to connect its receptors-mediated signaling with the CSC. See bioRxiv. 2020 (doi: https://doi.org/10.1101/2020.06.10.145003), bioRxiv 2020 (doi: https://doi.org/10.1101/2020.05.24.112847) and bioRxiv 2021 (doi: https://doi.org/10.1101/2021.05.24.445510).

As a sex steroid hormone, PRG exerts its cellular responses through signaling cascades involving classic (genomic), non-classic (non-genomic), or both combined responses by binding to either classic nuclear PRG receptors (nPRs, PR1/2) or non-classic membrane PRG receptors (mPRs, PAQRs). Using an nPR positive [nPR(+)] breast cancer cell line, T47D, Applicant demonstrated that PRG actions are realized through the balanced efforts between nPRs and mPRs signaling, and are further fine-tuned by the CSC (FIG. 2). See bioRxiv 2020 (doi: https://doi.org/10.1101/2020.05.24.112847) and bioRxiv 2021 (doi: https://doi.org/10.1101/2021.05.24.445510)

Without being bound by theory, Applicant envisions that a common mechanism exists among the CSC-PRG-mPRs signaling cascade under steroid actions, regardless of nPR(+) or (−) cell type. Applicant's key findings indicate that PRG and its antagonist, mifepristone (MIF), can work independently or synergistically to disrupt the CSC through their common targets, mPRs (FIG. 2). See bioRxiv. 2020 (doi: https://doi.org/10.1101/2020.06.10.145003), bioRxiv 2020 (doi: https://doi.org/10.1101/2020.05.24.112847) and bioRxiv 2021 (doi: https://doi.org/10.1101/2021.05.24.445510).

In regards to sex steroids in CCMs pathogenesis, CCMs are more common in women and become symptomatic during their reproductive period (the 30s-40s age range). Although no conclusive results have been found, hormonal changes during pregnancy have long been suggested as significant factors for increased bleeding, and female gender is a key risk factor for bleeding in CCM patients.

The increase in the size of CCM lesions and increased cases of hemorrhagic CCMs during pregnancy have been well documented, suggesting that pregnancy is associated with an increased risk of hemorrhage. It has been long speculated that the flux of hormones during pregnancy may predispose CCMs to hemorrhage. Significantly increased PRG levels during early pregnancy has been indicated to enhance the progression of lesions, possibly through its ability to induce structural changes within the vessels. Increased risk for acute CCM bleedings, or formation of a de novo CCM lesion have also been reported during pregnancy.

Collectively, these findings reinforce the idea that there are gender and sex hormone-associated differences in hemorrhagic stroke pathophysiology, and suggest that PRG-mediated signaling is a potential serum biomarker and molecular mediator in strokes. Currently, nPR(+) endothelial cells (ECs) can only be found in the veins and lymphatics of the uterus and ovary of the female reproductive system, where human umbilical vein endothelial cells (HUVECs) are derived. The vast majority of vascular ECs derived from other tissues are nPR(−) and mPRs(+), where only non-classic actions (via mPRs) of PRG on nPR(−) ECs have been reported.

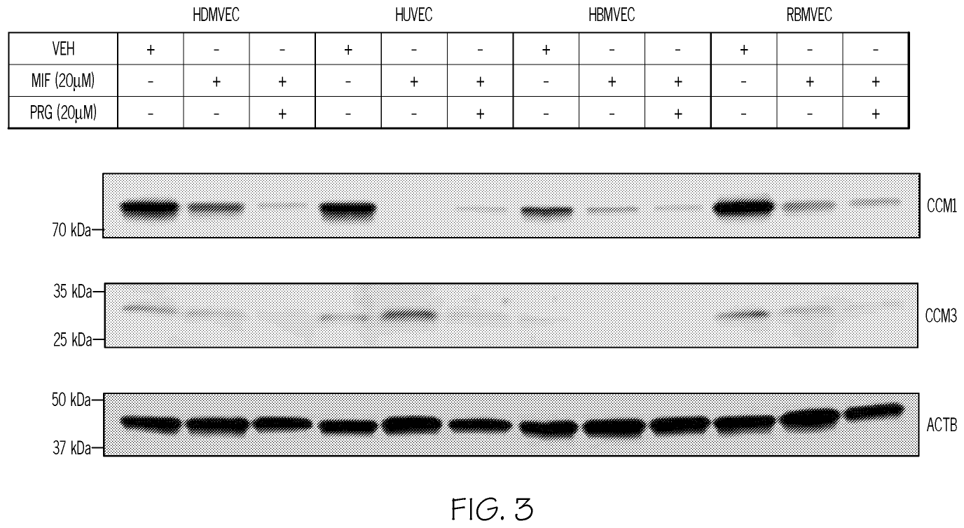

Applicant examined the CSC-mPRs complex modulating angiogenic signaling under combined steroid treatment (PRG+MIF) in nPR(−) ECs: human brain microvascular endothelial cells (HBMVECs), human dermal microvascular endothelial cells (HDMVECs), rat brain microvascular endothelial cells (RBMVECs), and nPR(+) ECs: HUVECs. Applicant's data support a common regulatory mechanism underlying the inhibitory effects of PRG/MIF on the CSC, independent of nPRs. However, the sex hormone inhibition of CCM1/3 proteins expression in ECs is more dramatic, reaffirming that steroid hormones have much stronger actions on the stability of the CSC through mPRs in nPR(−) microvascular ECs (FIG. 3).

PRG actions increased permeability of EC monolayer in-vitro and compromised BBB integrity in-vivo. The haploinsufficiency of CCM proteins in microvascular ECs is an essential step in the pathogenesis of CCM lesions, as demonstrated by in-vivo studies with zebrafish and Ccms mice models, but is insufficient to form lesions. Although the “two-hit” model, which creates a null condition in the lesion, can be used to explain familial CCM cases, it fails to account for sporadic forms of CCM, which make up to 80% of all CCM cases. Additional studies have demonstrated that haploinsufficiency and even the “null” condition of CCMs might be insufficient in initiating hemorrhagic events of CCM lesions. Since the “two-hit” model cannot alone explain CCM ruptures, there must be a molecular “trigger” that initiates the hemorrhagic events of CCM lesions.

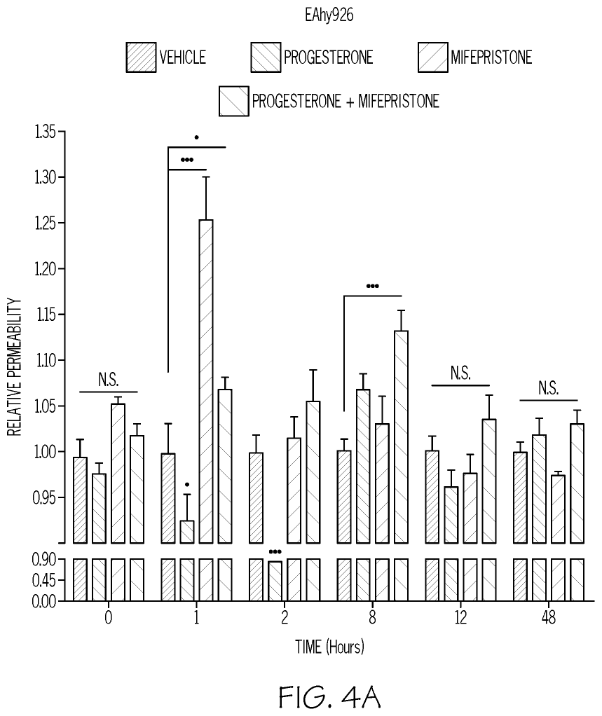

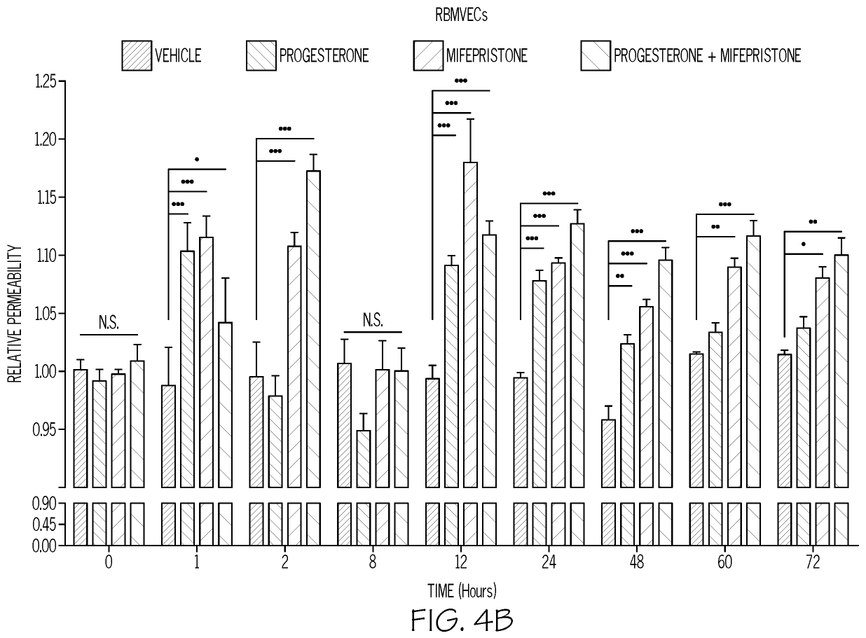

Based on Applicant's previous data, Applicant tested sex steroid actions on the in-vitro permeability of two different ECs lines, nPR(+) EAhy926 ECs derived from HUVECs and nPR(−) RBMVECs. Increased permeability was initially observed in both ECs, however, the permeability of nPR(+) EAhy926 ECs is back to normal after 12 hrs, while the permeability was continuously enhanced among all sex hormone treatments (PRG, MIF, PRG+MIF) of nPR(−) RBMVECs, indicating that permeability of nPR(−) ECs is more sensitive to steroid hormones via the CmP (CSC-mPRs-PRG) signaling axis (FIGS. 4A-4B). This finding was further supported by in-vivo permeability assays showing significantly increased BBB permeability of brain among all Ccms (1, 2, 3) mutants in the 90-day hormone treatment group, compared to WT mice.

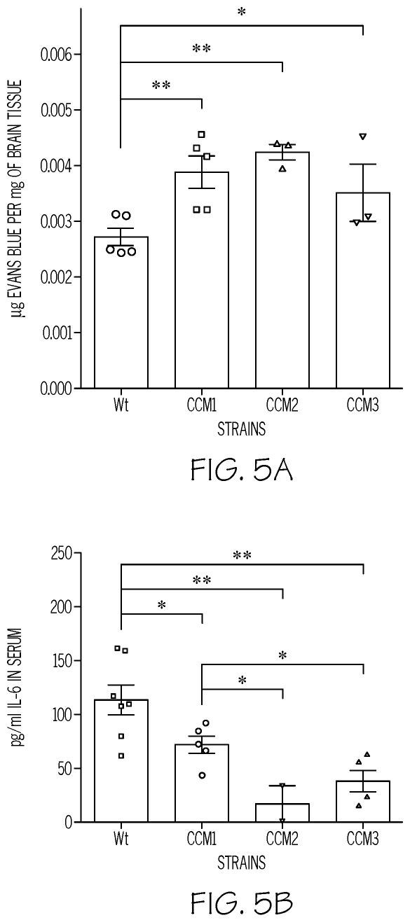

This concordant BBB leakage among all Ccms mutant mice was neither seen in other treatment groups, nor that of other organs (livers or lungs), indicating that chronic steroid actions specifically increases the BBB permeability of the brain, the primary location for CCM lesions (FIG. 5A). Therefore, BBB integrity among individuals with CCMs deficiency is susceptible to chronic exposure of excessive sex steroids.

### Example 1.4. Identification of Additional Biomarkers for Predicting Hemorrhagic Stroke

Disruption of PRG homeostasis lead to the discovery of the first set of biomarkers associated with BBB leakage Inflammatory cytokines have been on target as risk factors or biomarkers for cerebrovascular diseases and stroke, such as TNF-α, MCP-1, and IL-6. Many cytokines have been tested as prognostic or monitoring biomarkers to predict disease progression, risk assessment, outcome, and post-stroke management for ischemic strokes. Applicant identified immunosuppression in Ccms mutant mice, in which the levels of IL-6 were found to be inversely correlated with the progression of BBB leakage (FIG. 5B), and similar results observed in IL-12, suggesting that IL-6 and IL12 cytokines were suppressed under conditions of chronic steroid actions and reduced CCM expression.

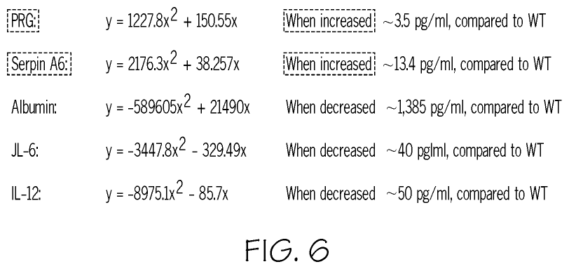

Additionally, Applicant's serum cytokine data in Ccms mutant mice have been supported by two separate pilot projects in two small human cohorts, further strengthening this proposal. Applicant performed regression with the serum concentration of the five biomarkers with the corresponding progression of compromised BBB competence for the same treatment groups to generate correlation functions (FIG. 6). Each biomarker has a unique cutoff value indicating an elevated risk of a hemorrhagic event.

In sum, Applicant has established that five serum etiological biomarkers (i.e., PRG, Albumin, Serpin A6, IL-6, and IL-12) are associated with disruption of the Blood-Brain Barrier (BBB), which could lead to hemorrhagic events. These findings indicate that the aforementioned biomarkers can be utilized to predict an onset of a cerebral hemorrhagic event in a subject.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of predicting an onset of a cerebral hemorrhagic event in a subject, said method comprising:
testing a blood sample of the subject for the presence or absence of one or more indicators, wherein the one or more indicators comprise:
an increased level of progesterone (PRG) relative to a reference blood sample,
a decreased level of Albumin relative to a reference blood sample,
an increased level of Serpin A6 relative to a reference blood sample,
a decreased level of IL-6 relative to a reference blood sample,
a decreased level of IL-12 relative to a reference blood sample, or
combinations thereof; and
correlating the presence or absence of the one or more indicators to the subject's risk for suffering from the cerebral hemorrhagic event,
wherein the presence of the one or more indicators is correlated to an increased risk in the subject for suffering from the cerebral hemorrhagic event, and
wherein the absence of the one or more indicators is correlated to a decreased risk in the subject for suffering from the cerebral hemorrhagic event.

2. The method of claim 1, further comprising a step of implementing a therapeutic decision to prevent the cerebral hemorrhagic event.

3. The method of claim 2, wherein the therapeutic decision comprises administering to the subject a therapeutic composition for preventing the hemorrhagic event.

4. The method of claim 2, wherein the therapeutic decision comprises evaluating the subject's brain for lesions and removing any identified lesions.

5. The method of claim 2, wherein the therapeutic decision is selected from the group consisting of increasing Albumin levels in a subject's blood, decreasing PRG levels in a subject's blood, decreasing ratios of PRG and estrogen (EST) in a subject's blood, decreasing Serpin A6 levels in a subject's blood, increasing IL-6 levels in a subject's blood, increasing IL-12 levels in a subject's blood, or combinations thereof.

6. The method of claim 1, wherein the cerebral hemorrhagic event comprises a hemorrhagic stroke.

7. The method of claim 1, wherein testing comprises testing for the presence or absence of a protein of the one or more indicators.

8. The method of claim 7, wherein the testing occurs through the utilization of an enzyme-linked immunosorbent assay (ELISA), an ultra-sensitive multiplex bead array assay (MBAA), or combinations thereof.

9. (canceled)

10. The method of claim 1, wherein the testing comprises testing for the presence or absence of a messenger RNA transcript of the one or more indicators.

11. The method of claim 1, further comprising a step of collecting the blood sample from the subject

12. The method of claim 1, wherein the blood sample is blood plasma.

13. The method of claim 1, wherein the blood sample is blood serum.

14. The method of claim 1, wherein:
the increased level of PRG is represented by an increase of at least 3.5 pg/ml relative to the reference blood sample,
the decreased level of Albumin is represented by a decrease of at least 1,385 pg/ml relative to the reference blood sample,
the increased level of Serpin A6 is represented by an increase of at least 13.4 pg/ml relative to the reference blood sample,
the decreased level of IL-6 is represented by a decrease of at least 40 pg/ml relative to the reference blood sample, and
the decreased level of IL-12 is represented by a decrease of at least 50 pg/ml relative to the reference blood sample.

15-18. (canceled)

19. The method of claim 1, wherein the subject is a human being.

20. The method of claim 1, wherein the one or more indicators comprise two or more of the following indicators:
an increased level of progesterone (PRG) relative to the reference blood sample,
a decreased level of Albumin relative to the reference blood sample,
an increased level of Serpin A6 relative to the reference blood sample,
a decreased level of IL-6 relative to the reference blood sample, and
a decreased level of IL-12 relative to the reference blood sample.

21. The method of claim 1, wherein the one or more indicators comprise three or more of the following indicators:
an increased level of progesterone (PRG) relative to the reference blood sample,
a decreased level of Albumin relative to the reference blood sample,
an increased level of Serpin A6 relative to the reference blood sample,
a decreased level of IL-6 relative to the reference blood sample, and
a decreased level of IL-12 relative to the reference blood sample.

22. The method of claim 1, wherein the one or more indicators comprise four or more of the following indicators:
an increased level of progesterone (PRG) relative to the reference blood sample,
a decreased level of Albumin relative to the reference blood sample,
an increased level of Serpin A6 relative to the reference blood sample,
a decreased level of IL-6 relative to the reference blood sample, and
a decreased level of IL-12 relative to the reference blood sample.

23. The method of claim 1, wherein the one or more indicators comprise:
an increased level of progesterone (PRG) relative to the reference blood sample,
a decreased level of Albumin relative to the reference blood sample,
an increased level of Serpin A6 relative to the reference blood sample,
a decreased level of IL-6 relative to the reference blood sample, and
a decreased level of IL-12 relative to the reference blood sample.

24. The method of claim 1, wherein the correlating occurs through the utilization of a software, wherein the software comprises a machine learning algorithm that correlates the presence or absence of the one or more indicators to the subject's risk for suffering from the cerebral hemorrhagic event.

25. (canceled)

26. The method of claim 1, wherein the subject's risk for suffering from the cerebral hemorrhagic event is characterized by at least a likelihood of progression of the cerebral hemorrhagic event, a likelihood of recurrence of the cerebral hemorrhagic event, a likelihood of regression of the cerebral hemorrhagic event, or combinations thereof.

27-40. (canceled)
